(19) United States
(12) Patent Application Publication
(10) Pub. No.: US 2021/0228495 A1
(43) Pub. Date: Jul. 29, 2021

(54) DRUG-LOADED TISSUE ADHESIVE FILM AND PREPARATION METHOD THEREFOR

(71) Applicant: SUZHOU BOCHUANG TONGKANG PHARMACEUTICAL TECHNOLOGY CO., LTD., KUNSHAN CITY, JIANGSU, (CN)

(72) Inventors: GUANGWAN LIU, KUNSHAN CITY, JIANGSU (CN); CHANGLIN WU, KUNSHAN CITY, JIANGSU (CN)

(73) Assignee: SUZHOU BOCHUANG TONGKANG PHARMACEUTICAL TECHNOLOGY CO., LTD., KUNSHAN CITY, JIANGSU (CN)

(21) Appl. No.: 16/077,722

(22) PCT Filed: Aug. 5, 2016
(86) PCT No.: PCT/CN2016/093401

§ 371 Date: Apr. 16, 2021

(30) Foreign Application Priority Data
Mar. 14, 2016 (CN) 2016101432887
May 31, 2016 (CN) 2016103783924

(51) Int. Cl. A61K 9/70; A61K 9/127; A61K 47/36; A61K 47/42; A61K 31/44; A61K 31/404; A61K 31/7032; A61K 38/20; A61K 31/517; A61K 31/506; A61K 31/4439; A61K 31/513; C07K 16/32; A61K 31/536; A61K 38/18; A61K 31/727; A61K 31/715; C12N 15/113; B05D 7/00

(52) U.S. Cl. CPC A61K 9/7046; A61K 2039/505; A61K 47/36; A61K 47/42; A61K 31/44; A61K 31/404; A61K 31/7032; A61K 38/2013; A61K 31/517; A61K 31/506; A61K 31/4439; A61K 31/513; C07K 16/32; A61K 31/536; A61K 38/1866; A61K 38/1808; A61K 38/185; A61K 38/1825; A61K 31/727; A61K 31/715; C12N 15/113; B05D 7/58; C07K 2317/24; A61K 9/127

(57) ABSTRACT

The present invention provides a drug-loaded tissue adhesive film, comprising alternately superposed cationic layers and anionic layers, at least one of the cationic layers and the anionic layers being a drug layer, or at least one of the cationic layers and the anionic layers containing a drug with charges. The provided drug-loaded tissue adhesive film has good tissue adhesiveness, biocompatibility, degradable absorption, and stability, and the physical and chemical properties of the drug-loaded tissue adhesive film can be adjusted by adjusting material compositions.

### FIELD OF INVENTION

The present invention relates to the field of biomedicines, and in particular, to a drug-loaded tissue adhesive film and a preparation method therefor. The drug-loaded tissue adhesive film may be implanted into an infected tissue part through a surgery to realize local direct slow-release and controlled-release drug delivery to treat various diseases.

### BACKGROUND

With rapid development of science and technology, a large number of drugs of new types have been discovered, but the biggest obstacle that prevents many new drugs from clinical use is the lack of effective drug delivery technology. For example, nucleic acid drugs are easily degraded by nucleases, which results in not reaching targets or entering the interior of cells. Specific examples of nucleic acid drugs are RNA interference. RNA interference is a double-stranded small interfering RNA-mediated gene silencing technology consisting of about twenty nucleotides, which is sequence-specific, and thus small nucleic acid based on RNA interference has great application prospects in diseases treatment. However, since small nucleic acid molecules including siRNA do not have the ability to target tissues or cells, the ability to penetrate cell membranes is poor, and it is extremely unstable in physiological environments. So, the drug delivery system, especially the carrier, is a key problem that needs to be solved urgently, which is one of the key factors for the successful application of small nucleic acid drugs in clinical application. For other drugs, there are various problems that need to be improved in terms of carriers, therefore research on drug delivery systems, especially carriers, has received increasing attention.

### SUMMARY

In view of the above-mentioned disadvantages of the prior art, the purpose of the present invention is to provide a drug-load tissue adhesive film, a method for preparing the same and application thereof. The drug-loaded tissue adhesive film can be implanted into an infected tissue part through a surgery to realize local direct slow-release and controlled-release drug delivery to treat various diseases, and is used for solving the problem in the prior art.

In order to realize the above-mentioned and other related purposes, in one aspect, the present invention provides a drug-loaded tissue adhesive film, the drug-loaded tissue adhesive film comprises alternately superposed cationic layers and anionic layers, at least one of the cationic layers and the anionic layers is a drug layer, or at least one of the cationic layers and the anionic layers contains a drug with charges.

Specifically, the cationic layer presents positive charges on the whole and the anionic layer presents negative charges on the whole.

When at least one of the cationic layers and the anionic layers is the drug layer, the cationic layer and/or the anionic layer used as the drug layer is the drug with charges, the cationic layer not used as the drug layer may contain a carrier material with a cationic group and the anionic layer not used as the drug layer may contain a carrier material with an anionic group.

When at least one of the cationic layers and the anionic layers contains the drug with charges, the cationic layer may contain a carrier material with a cationic group and the anionic layer may contain a carrier material with an anionic group.

In the drug-loaded tissue adhesive film provided by the present invention, the carrier material with a cationic group is a material with positive charges itself or a material with positive charges after ionization in solvent. In one embodiment of the present invention, the carrier material may be a substance with positive charges after ionization in water.

The carrier material with a cationic group is preferably a biocompatible material that is degradable in vivo.

Specifically, the carrier material with a cationic group is one or a combination of an organic high-molecular polymer with a cationic group, a polysaccharide with a cationic group, a polypeptide with a cationic group, a protein with a cationic group and a cationic liposome.

The organic high-molecular polymer specifically refers to a high-molecular-weight compound formed by many identical structural units (one structural unit or more structure units) through repetitive connection of covalent bonds. In one embodiment of the present invention, the specifically selectable organic high-molecular polymer with a cationic group includes but is not limited to one or a combination of polyethyleneimine, polyamidoamine dendrimer, cationic polyester (specifically examples are cationic polyphosphoester, polyphosphorylester, polymethacrylate, etc.) and polyvinyl pyridine.

The polysaccharide specifically refers to a saccharide which can form a plurality of monosaccharides, more specifically ten or more monosaccharides, when the molecule is hydrolyzed, and the polysaccharide with a cationic group can be a polysaccharide with a cationic group itself, and may also be a polysaccharide with a cationic group obtained by modification. The technique of modifying a polysaccharide to have a cationic group is a prior art in the field, and specifically may be a method of grafting an amino group on a side chain. In one embodiment of the present invention, the specifically selectable polysaccharide with a cationic group includes but is not limited to chitosan and derivatives thereof (specific examples are chitosan quaternary ammonium salt, low-substituted carboxymethyl chitosan), trimethyl chitosan, imidazolyl-containing chitosan, thiolated chitosan, etc.), cationic starches and derivatives (specific examples are cyclodextrin with a cationic group, etc.) and one or a combination of other polysaccharides with a cationic group.

The polypeptide or protein with a cationic group may be a polypeptide or protein with a cationic group itself, and may also be a polypeptide or protein with a cationic group obtained by modification. In one embodiment of the invention, the specifically selectable polypeptide or protein with a cationic group includes but is not limited to one or a combination of more of polylysine or polyarginine and derivatives thereof (specific examples of derivatives are polypeptide to which PEG is introduced, galactose, lactose, folic acid, transferrin, etc.), collagen, gelatin and serum albumin. Since some proteins (such as collagen, gelatin and serum albumin) may present positive charges or negative charges at different pH values (positive charges at pH below isoelectric point, and negative charges at pH above isoelectric point), this type of substance can be used as a cationic layer or an anionic layer.

The cationic liposome can be selected from various cationic liposomes in the field, and specific examples include but are not limited to cationic liposomes prepared using DOTMA analogs, DOTAP, spermidine cholesterol.

In the drug-loaded tissue adhesive film provided by the present invention, the carrier material with a cationic group is a material with negative charges itself or a material with negative charges after ionization in solvent and. In one embodiment of the present invention, the carrier material may be a substance with negative charges after ionization in water and n.

The carrier material with an anionic group is preferably a biocompatible material that is degradable in vivo.

Specifically, the carrier material with an anionic group is one or a combination of an organic high-molecular polymer with an anionic group, a polysaccharide with an anionic group, a polypeptide with an anionic group, a protein with an anionic group, and an anionic liposome.

In one embodiment of the present invention, the specifically selectable organic high-molecular polymer with an anionic group includes but is not limited to an anion composed of dicarboxylic acid as given in CN1524184A.

The polysaccharide with an anionic group may be a polysaccharide with an anionic group itself, or a polysaccharide with an anionic group obtained by modification. The technique of modifying a polysaccharide to have an anionic group a prior art in the field, and specifically it may be anionic starch (more specific examples are carboxymethyl starches). In one embodiment of the present invention, the specifically selectable polysaccharide with an anionic group includes but is not limited to one or a combination of carboxymethyl cellulose, carboxymethyl chitosan, hyaluronic acid, alginic acid, carboxymethyl starch, chondroitin sulfate, heparin and derivatives thereof, and other polysaccharides with an anionic group.

The polypeptide with an anionic group may be a polypeptide with an anionic group itself, or a polypeptide with an anionic group obtained by modification. In one embodiment of the present invention, the specifically selectable polypeptide with an anionic group includes but is not limited to one or a combination of polyglutamic acid or polyaspartic acid and derivatives thereof, collagen and gelatin.

The anionic liposome can be selected from various anionic liposomes in the field, and specific examples include but are not limited to AS-ODNs anionic liposomes and DOPG/DOPE anionic liposomes.

In the drug-loaded tissue adhesive film provided by the present invention, the drug with charges may specifically be a drug with positive charges and/or a drug with negative charges, and as long as it carries charges, it can be used as a drug layer or contained in the drug-loaded tissue adhesive film, which is generally included in an amount effective to treat. The therapeutically effective amount corresponds to the purpose of the therapeutic indication, and specifically refers to the effect that a therapeutic amount can achieve a therapeutic indication after an appropriate drug delivery period. The treatment specifically includes prophylactic, curative or palliative treatment of pharmaceutical and/or physiological effects. Preferably, the effect refers to medically reducing one or more symptoms of the indication or completely eliminating the indication, or retarding, delaying the occurrence of the indication and/or reducing the risk of developing or worsening the indication.

Specifically, when at least one of the cationic layers and the anionic layers is the drug layer, the drug layer may be specifically selected to be a drug with charges. Specifically, when the cationic layer is the drug layer, it may comprise a drug with positive charges; and when the anionic layer is the drug layer, it may comprise a drug with negative charges.

More specifically, for the drug with positive charges, when at least one of the cationic layers is the drug layer, the drug with positive charges may be used as the drug layer and form an electrostatic effect with an adjacent anionic layer; and when it is used as a drug contained in the cationic layer and/or the anionic layer, it may be located in the cationic layer and form an electrostatic effect with an adjacent anionic layer, such that it is stably contained in the drug-loaded tissue adhesive film or is capable of being located in the anionic layer, and after the drug with positive charges acts with the carrier material with an anionic group, this layer still presents negative charges on the whole and forms an electrostatic effect with the carrier material with a cationic group, such that it is stably contained in the drug-loaded tissue adhesive film. For the drug with negative charges, when at least one of the anionic layers is the drug layer, the drug with negative charges may be used as the drug layer and form an electrostatic effect with an adjacent cationic layer; and when it is used as a drug contained in the cationic layer and/or the anionic layer, it may be located in the anionic layer and form an electrostatic effect with an adjacent cationic layer, such that it is stably contained in the drug-loaded tissue adhesive film or is capable of being located in the material buffer layer with a cationic group, and after the drug with negative charges acts with the carrier material with a cationic group, this layer still presents positive charges on the whole and forms an electrostatic effect with the carrier material with an anionic group, such that it is stably contained in the drug-loaded tissue adhesive film.

The drugs with charges may be drugs with charges itself, or drug complexes with charges formed by some drugs which are combined or coated with substances or materials with charges (positive charges or negative charges). A method for forming drug complexes with charges by drugs which are combined or coated with substances or materials with charges (positive charges or negative charges) is a prior art in the field, and specific drug complexes include but are not limited to drug liposomes, polymers formed by drugs and anionic group materials, and polymers formed by drugs and cationic group materials. Forms of drug complexes may be microspheres, microcapsules, microparticles, micromasses and micelles.

Specifically, the drug includes but is not limited to one or a combination of nucleic acid drugs, polypeptide drugs, protein drugs, polysaccharide, compound drugs, lipid drugs and derivatives thereof.

The nucleic acid drugs include nucleic acids or derivatives thereof. The nucleic acids specifically include but are not limited to siRNA, microRNA, mRNA, tRNA, rRNA, RNA viruses, ribozyme, antisense nucleic acid, peptide nucleic acid, triple-stranded nucleic acid, DNA plasmids, DNA viruses and DNA protein synthetic genes. Various nucleic acids may be usedseparately, or used by combining more than two nucleic acids properly. The nucleic acids may be nucleic acids coming from human, animals, plants, bacteria, viruses and the like, or nucleic acids prepared through chemical analysis. Therefore, the nucleic acids may be one of single-stranded, dual-stranded and triple-stranded nucleic acids, and there is no special limitation to the molecular weight thereof.

The polypeptide drugs include polypeptides or derivatives thereof, and specifically include but are not limited to cell targeting polypeptides (specifically such as polypeptides containing arginine-glycine-aspartic acid, valine-glycine-valine-alanine-proline-glycine and isoleucine-lysine-valine-alanine-valine segments) and antibacterial peptides.

The protein drugs include proteins or derivatives thereof, and specifically include but are not limited to cytokine, antibodies, ligands, glycoproteins, coagulation factors and interferons.

The polysaccharide drugs include polysaccharides or derivatives thereof, and specifically include but are not limited to proteoglycan, heparin, small molecule heparin, fructose phosphate and lentinan.

The compound drugs generally refer to compounds having a single structure and more specifically small molecule compound drugs, and the molecular weight thereof is generally not greater than 20000, preferably not greater than 15000, more preferably not greater than 10000, more preferably not greater than 8000, more preferably not greater than 6000, more preferably not greater than 4000, more preferably not greater than 3000, more preferably not greater than 2000, more preferably not greater than 1500, and further preferably not greater than 1000. The compound drugs may be chemically synthesized, and may also be compounds separated and extracted from plants, animals and microorganisms. Specifically, the compound drugs include but are not limited to fluorouracil, Sunitinib, Sorafenib, camptothecin, paclitaxel, rapamycin and etoposide, and also include antibiotics (e.g., colistin, ofloxacin, amoxicillin, clarithromycin, cefazolin, etc.), antiviral drugs (entecavir, adenosine, dideoxythymidine, polyt: C, azidothymidine, acyclovir, amantadine, bromovinyl uridine, etc.), and the compound drugs may be compound drugs with charges and may be drug complexes with charges formed by compound drugs without charges and substances with charges, such as Sorafenib microspheres coated with sodium alginate.

The lipid drugs include but are not limited to gangliosides.

In the drug-loaded tissue adhesive film provided by the present invention, the cationic layer and the anionic layer are alternately superposed, and can be combined by electrostatic attraction to form a tissue adhesive film for loading the drug, the cationic layer presents positive charges on the whole, the anionic layer presents negative charges on the whole. Specifically, it may be a method in which a cationic layer material and an anionic layer material are alternately deposited by electrostatic attraction, such as a polyelectrolyte layer-by-layer self-assembly method. The number of times of alternately superposing the cationic layers and the anionic layers is not particularly limited, and one skilled in the art can adjust the total thickness of the tissue adhesive film according to actual needs (e.g., drug loading rate, degradation time), and the total thickness of the tissue adhesive film may be smaller than or equal to 1000 μm, more specifically smaller than or equal to 600 μm, more specifically smaller than or equal to 400 μm, more specifically smaller than or equal to 200 μm, and further specifically may be 0.05 μm-1000 μm. For each of cationic layers and/or anionic layers, one skilled in the art may adjust the thickness of each cationic layer and/or anionic layer according to the type of the material and the actual need (e.g., drug loading rate and degradation time), and can further determine the use amount of the drug, the carrier material with a cationic group and the carrier material with an anionic material, the thickness thereof may be 0.0005 μm-100 μm, more specifically 0.001 μm-50 μm, more specifically at a nanometer level, and the ratio of the use amount of the material in the preparation of the cationic layers to the use amount of the material in the preparation of the anionic layers is 1:0.005-200.

In a second aspect, the present invention provides a method for preparing a drug-loaded tissue adhesive film, comprising the following step: alternately depositing cationic layers and anionic layers on a substrate to prepare the tissue adhesive film.

The drug-loaded tissue adhesive film provided by the invention can be completely peeled off from the substrate after being prepared, and can stably exist independent of other adhesive materials (such as the substrate or the backing).

Specifically, when at least one of the cationic layers and the anionic layer is a drug layer, the preparation method may be a polyelectrolyte layer-by-layer self-assembly method, and specifically comprise the following steps:

alternately depositing cationic layers and anionic layers on the substrate to prepare the tissue adhesive film, wherein a drug with charges is used when a material layer is deposited as a drug layer.

Specifically, the method of alternately depositing the cationic layers and the anionic layers on the substrate specifically includes the following steps: when the non-drug layer is deposited, enabling the substrate to be alternately immersed in or coated with the solution of the carrier material with a cationic group and the solution of the carrier material with an anionic group; and when the drug layer is deposited, enabling the substrate to be alternately immersed in or coated with the solution of the drug with charges, washing and drying after each time of deposition (specifically, washing with water), and peeling off the film after the alternate deposition is completed, to obtain the tissue adhesive film.

More specifically, the solution of the material with a cationic group, the solution of the material with an anionic group and the solution of the drug with charges may be aqueous solution, appropriate salt ions may be added to the aqueous solution to change the spatial extension of the molecule, and the type of salt ions that can be specifically used to change the spatial extension of the molecule is known in the prior art, such as NaCl, KCl added to the solution.

One skilled in the art can adjust the concentration, the pH value, the salt ion concentration and the like of the solution used in the deposition according to the type of the material. In one embodiment of the present invention, the salt ion concentration in the solution used for deposition may be specifically smaller than or equal to 10 mol/L, more specifically 0.05-0 mol/L, and the pH value may be 3-11.

When at least one of the cationic layers and the anionic layer contains a drug with charges, the preparation method may be a polyelectrolyte layer-by-layer self-assembly method, which comprises the following steps:

alternately depositing the cation layers and the anion layers on the substrate to prepare the tissue adhesive film; and in the process of preparing the tissue adhesive film, the drug is implanted into the tissue adhesive film.

Specifically, the method of alternately depositing the cationic layer and the anionic layer on the substrate specifically includes the following steps: enabling the substrate to be alternately immersed in or coated with solution of the carrier material with a cationic group and solution of the carrier material with an anionic group (when a material comprising a drug is deposited, the solution for depositing the material layer may further comprise the drug), washing and drying after each time of deposition (specifically washing with water), and peeling off the film after the alternate deposition, to obtain the tissue adhesive film.

One skilled in the art may select a suitable method for implanting a drug into the tissue adhesive film. Specifically, when the cationic layer containing the drug with charges is deposited, the drug may be mixed with the carrier material with a cationic group for deposition. (specifically enabling the carrier material with a cationic group to be immersed in or coated with mixed solution of the carrier material with a cationic group and the drug); when the anionic layer containing the drug with charges is deposited, the drug is mixed with the carrier material with an anionic group for deposition (specifically, enabling the carrier material with an anionic group to be immersed in or coated with mixed solution of the carrier material with an anionic group and the drug); and in addition, after the tissue adhesive film is prepared, the solution containing the drug is instantaneously coated at high pressure, and further washing and drying are performed.

In a third aspect, the present invention provides application of a tissue adhesive film to preparation of a drug carrier material.

The drug is specifically a drug with charges.

Specifically, the tissue adhesive film comprises alternately superposed cationic layers and anionic layers.

More specifically, at least one of the cationic layers and the anionic layers is a drug layer, or at least one of the cationic layers and the anionic layers is used for containing a drug with charges.

The drug-loaded tissue adhesive film provided by the present invention has good tissue adhesion, good biocompatibility and degradable absorption, good stability, and physical and chemical properties thereof can be adjusted by adjusting the composition of the material. Besides, the method for preparing the drug-loaded tissue adhesive film is simple, the drug loading rate can be adjusted according to the required amount, the carrier material can protect the carried drug (such as protecting the RNA from being degraded), and can transfer the carried drug efficiently into the target cells, for example, the drug-loaded tissue adhesive film can be directly adhered to the lesion site through a surgery (i.e., treating various diseases through surgical implantable slow-release drug delivery), and the drug is delivered directly, the targeting ability is good, the local drug concentration is high, the therapeutic effect is good, the side effect is small, the potential toxicity to organisms is low and the bio-safety is very high.

### BRIEF DESCRIPTION OF THE DRAWINGS

FIG. 1 illustrates a schematic view in a gel electrophoresis experiment of siRNA in embodiment 12.

FIG. 2 illustrates schematic views of cell transfection tests in embodiment 13.

FIG. 3 illustrates a schematic view of relative fluorescence intensity in embodiment 13.

### DETAILED DESCRIPTION OF THE PREFERRED EMBODIMENTS

The embodiments of the present invention will be described below through specific examples. One skilled in the art can easily understand other advantages and effects of the present invention according to the content disclosed in the description. The present invention may also be implemented or applied through other different specific implementation modes. Various modifications or variations may be made to all details in the description based on different points of view and applications without departing from the spirit of the present invention.

Before the embodiments of the present invention are further described, it should be understood that the protective scope of the present invention is not limited to the following specific implementation solutions; and it should be further understood that the terms used in the embodiments of the present invention are used for describing specific implementation solutions instead of limiting the protective scope of the present invention; and in the description and claims of the present invention, unless otherwise clearly pointed out, the singular forms “a,” “an,” and “the” are intended to include the plural forms as well.

When numerical value ranges are given in the embodiments, it should be understood that, unless otherwise indicated, two endpoints of each numerical value range and any numerical values between the two endpoints are selectable. Unless otherwise defined, all technical and scientific terms used in the present invention have the same meaning as commonly understood by one skilled in the art. In addition to the specific methods, devices and materials used in the embodiments, as known by one skilled in the art to the prior art and recorded in the present invention, any methods, devices and materials in the prior art similar or equal to the methods, devices and materials in the embodiments of the present invention may also be used to implement the present invention.

Unless otherwise stated, the experiment methods, detection methods and preparation methods disclosed in the present invention adopt conventional molecular biology, biochemistry, chromatin structure and analysis, analytical chemistry, cell cure, recombinant DNA technique and common techniques in the related art. These techniques have already been perfectly described in the current literatures. For details, refer to Sambrook, et al, MOLECULAR CLONING: A LABORATORY MANUAL, Second edition, Cold Spring Harbor Laboratory Press, 1989 and Third edition, 2001; Ausubel, et al, CURRENT PROTOCOLS IN MOLECULAR BIOLOGY, John Wiley & Sons, New York, 1987 and periodic updates; the series METHODS IN ENZYMOLOGY, Academic Press, San Diego; Wolfe, CHROMATIN STRUCTURE AND FUNCTION, Third edition, Academic Press, San Diego, 1998; METHODS IN ENZYMOLOGY, Vol. 304, Chromatin (P. M. Wassarman and A. P. Wolfe, eds.), Academic Press, San Diego, 1999; and METHODS IN MOLECULAR BIOLOGY, Vol. 119, Chromatin Protocols (P. B. Becker, ed.), Humana Press, Totowa, 1999, etc.

### Embodiment 1

In an aseptic bench, a culture dish with diameter of 12 cm was prepared, a silicon dice film with the same diameter was put therein (subjected to washing, sterilization and depyrogenation treatment), and drying was performed; solution A of a material with a cationic group (1 mg/ml chitosan, 0.1 mol/L acetic acid, 0.2 mol/L NaCl, pH=4) and solution B of a material with an anionic group (1 mg/ml carboxymethyl chitosan, 0.15 mol/L NaCl, pH=6) were respectively prepared; part of solution B was taken and added with a small interfering nucleic acid drug (eGFP-siRNA (sense: 5′-GGCACAAGCUGGAGUACAAUU-3′; antisense: 5′-UUGUACUCCAGCUUGUGCCUU-3′, 20 ug/ml), a cell targeting factor (hyaluronic acid with a molecular weight smaller than 20000 Daltons, 10 ug/ml) and a targeting polypeptide (1*10⁻⁴ mg/ml) to prepare solution C, wherein an amino acid sequence of the targeting polypeptide was: valine-glycine-valine-alanine-proline-glycine; the solution was respectively filled into a high-pressure instantaneous coating device, firstly the solution C was instantaneously coated at high pressure into the culture dish, and drying was performed to form a film; then the solution A was instantaneously coated at high pressure and washing was performed by the water for injection; then the solution B was instantaneously coated at high pressure, washing was performed by water for injection, the solution A and the solution B were alternately coated in this way repeatedly; then the solution A was instantaneously coated at high pressure and washing was performed by water for injection; and finally the solution C was instantaneously coated at high pressure, washing was performed by using water for injection, drying was performed, the film was peeled off, an adhesive side of the film was washed by using water for injection, and then dried to obtain an implantable tissue adhesive film loading the small interfering nucleic acid drug.

### Embodiment 2

In an aseptic bench, a high-molecular material plate was provided (subjected to washing, sterilization and depyrogenation treatment), and drying was performed; solution A of a material with a cationic group (2 mg/ml chitosan, 0.1 mol/L acetic acid, 0.2 mol/L NaCl, pH=4) and solution B of a material with an anionic group (2 mg/ml alginic acid with a molecular weight of 60000-80000, 0.15 mol/L NaCl, pH=6) were respectively prepared; the solution B was added with a small interfering nucleic acid drug (the same as embodiment 1, 20 ug/ml) and a cell targeting factor (hyaluronic acid with a molecular weight smaller than 20000 Daltons, 10 ug/ml), and the solution A was added with a targeting polypeptide (1*10⁻⁴ mg/ml), wherein an amino acid sequence of the targeting polypeptide was: isoleucine-lysine-valine-alanine-valine; the plate was firstly immersed in the solution A for 20 min, taken out, put in washing solution for washing and dried; and then the plate was immersed in the solution B for 30 min, taken out, put in washing solution for washing and dried, the operations were alternately performed for 160 times in this way, finally washing was performed by using water for injection, drying was performed and the film was peeled off to obtain an implantable tissue adhesive film loading the small interfering nucleic acid drug.

### Embodiment 3

In an aseptic bench, a high-molecular material plate was provided (subjected to washing, sterilization and depyrogenation treatment), and drying was performed; solution A of a material with a cationic group (2 mg/ml chitosan, 0.1 mol/L acetic acid, 0.2 mol/L NaCl, pH=4) and solution B of a material with an anionic group (2 mg/ml hyaluronic acid, 0.15 mol/L NaCl, pH=6) were respectively prepared; the solution A was added with a small interfering nucleic acid cationic liposome (for a method for preparing a cationic liposome, please refer to Anionic Liposome-Cationic Liposome Complex Mediated Gene Transfection, Journal of Pharmaceutical Practice, 2011 (29): 4, the small interfering nucleic acid is the same as that in embodiment 1, the added amount of the small interfering nucleic acid cationic liposome is 0.5 mg/ml, the drug loading rate of the small interfering nucleic acid is 21.7%), and uniform stirring with a targeting polypeptide (1*10⁻⁴ mg/ml) was performed, wherein an amino acid sequence of the targeting polypeptide was: arginine-glycine-aspartic acid; the plate was firstly immersed in the solution A for 20 min, taken out, put in washing solution for washing and dried; and then the plate was immersed in the solution B for 30 min, taken out, put in washing solution for washing and dried, the operations were alternately performed for 180 times in this way, finally washing was performed by using water for injection, drying was performed and the film was peeled off to obtain an implantable tissue adhesive film loading the small interfering nucleic acid drug.

### Embodiment 4

In an aseptic bench, a high-molecular material plate was provided (subjected to washing, sterilization and depyrogenation treatment), and drying was performed; solution A of a material with a cationic group (2 mg/ml chitosan, 0.1 mol/L acetic acid, 0.2 mol/L NaCl, pH=4) and solution B of a material with an anionic group (2 mg/ml hyaluronic acid, 0.15 mol/L NaCl, pH=6) were respectively prepared; the solution B was added with a small interfering nucleic acid anionic liposome (for a preparation method, please refer to Anionic Liposome-Cationic Liposome Complex Mediated Gene Transfection, Journal of Pharmaceutical Practice, 2011 (29): 4, the small interfering nucleic acid is the same as that in embodiment 1, the added amount of the small interfering nucleic acid anionic liposome is 0.5 mg/ml, the drug loading rate of the small interfering nucleic acid is 12.4%), uniform stirring with a targeting polypeptide (1*10⁻⁴ mg/ml) was performed, wherein an amino acid sequence of the targeting polypeptide was: valine-glycine-valine-alanine-proline-glycine; the plate was firstly immersed in the solution A for 20 min, taken out, put in washing solution for washing and dried; and then the plate was immersed in the solution B for 30 min, taken out, put in washing solution for washing and dried, the operations were alternately performed for 180 times in this way, finally washing was performed by using water for injection, drying was performed and the film was peeled off to obtain an implantable tissue adhesive film loading the small interfering nucleic acid drug.

### Embodiment 5

In an aseptic bench, a culture dish with diameter of 12 cm was provided, a high-molecular material film with the same diameter was put therein (subjected to washing, sterilization and depyrogenation treatment), washing was performed by using water for injection and drying was performed; solution A of a material with a cationic group (1 mg/ml collagen, 0.1 mol/L acetic acid, 0.2 mol/L NaCl, pH=3.5) and solution B of a material with an anionic group (1 mg/ml hyaluronic acid, 0.15 mol/L NaCl, pH=6) were respectively prepared; the solution A was added with a small interfering nucleic acid cationic liposome (for a preparation method, refer to embodiment 3, the concentration being 0.5 mg/ml) and a targeting polypeptide (1*10⁻⁴ mg/ml), and uniform stirring was performed, wherein an amino acid sequence of the targeting polypeptide was: arginine-glycine-aspartic acid; the solution was respectively filled into a high-pressure instantaneous coating device, firstly the solution A was instantaneously coated at high pressure into the culture dish, drying was performed, then the solution B was instantaneously coated at high pressure, washing was performed by using water for injection, the solution A and the solution B were alternately coated in this way repeatedly for 200 times; and finally the solution A was instantaneously coated at high pressure, washing was performed by using water for injection, drying was performed, the film was peeled off, an adhesive side of the film was washed by using water for injection, and dried to obtain an implantable tissue adhesive film loading the small interfering nucleic acid drug.

### Embodiment 6

In an aseptic bench, a high-molecular material plate was provided (subjected to washing, sterilization and depyrogenation treatment), and drying was performed; solution A of a material with a cationic group (2 mg/ml chitosan, 0.1 mol/L acetic acid, 0.2 mol/L NaCl, pH=4) and solution B of a material with an anionic group (2 mg/ml alginic acid with a molecular weight of 60000-80000, 0.15 mol/L NaCl, pH=6) were respectively prepared; the solution B was added with a Sorafenib hyaluronic acid microsphere (the concentration is 1.5 mg/ml, the diameter of the microsphere is 10-16 μm, the drug loading rate is 7.58%), and the solution A was added with a targeting polypeptide (1*10⁻⁴ mg/ml), wherein an amino acid sequence of the targeting polypeptide was: isoleucine-lysine-valine-alanine-valine; the plate was firstly immersed in the solution A for 20 min, taken out, put in washing solution for washing and dried; and then the plate was immersed in the solution B for 30 min, taken out, put in washing solution for washing and dried, the operations were alternately performed for 30 times in this way, finally washing was performed by using water for injection, drying was performed and the film was peeled off to obtain an implantable tissue adhesive film loading the Sorafenib drug.

### Embodiment 7

In an aseptic bench, a culture dish with diameter of 12 cm was provided, a high-molecular material film with the same diameter was put therein (subjected to washing, sterilization and depyrogenation treatment), washing was performed by using water for injection and drying was performed; solution A of a material with a cationic group (1 mg/ml collagen, 0.1 mol/L acetic acid, 0.2 mol/L NaCl, pH=3.5) and solution B of a material with an anionic group (1 mg/ml hyaluronic acid, 0.15 mol/L NaCl, pH=6) were respectively prepared; the solution B was added with a Sunitinib sodium alginate microsphere (the concentration is 1.0 mg/ml, the diameter of the microsphere is 10-15 μm, the drug loading rate is 6.28%), the solution A was then added with a targeting polypeptide (1*10⁻⁴ mg/ml) and uniform stirring was performed, wherein an amino acid sequence of the targeting polypeptide was: arginine-glycine-aspartic acid; the solution was respectively filled into a high-pressure instantaneous coating device, firstly the solution A was instantaneously coated at high pressure into the culture dish, drying was performed, then the solution B was instantaneously coated at high pressure, washing was performed by using water for injection, the solution A and the solution B were alternately coated in this way repeatedly for 50 times; and finally the solution A was instantaneously coated at high pressure, washing was performed by using water for injection, drying was performed, the film was peeled off, an adhesive side of the film was washed by using water for injection, and dried to obtain an implantable tissue adhesive film loading the Sunitinib drug.

### Embodiment 8

In an aseptic bench, a high-molecular material plate was provided (subjected to washing, sterilization and depyrogenation treatment), and drying was performed; solution A of a material with a cationic group (2 mg/ml chitosan, 0.1 mol/L acetic acid, 0.2 mol/L NaCl, pH=4) and solution B of a material with an anionic group (2 mg/ml hyaluronic acid, 0.15 mol/L NaCl, pH=6) were respectively prepared; the solution B was added with a ganglioside sodium alginate microsphere (the concentration is 1.0 mg/ml, the diameter of the microsphere is 0.15-0.26 μm, the drug loading rate being 7.3%), and uniform stirring with a targeting polypeptide (1*10⁻⁴ mg/ml) was performed, wherein an amino acid sequence of the targeting polypeptide was: valine-glycine-valine-alanine-proline-glycine; the plate was firstly immersed in the solution A for 20 min, taken out, put in washing solution for washing and dried; and then the plate was immersed in the solution B for 30 min, taken out, put in washing solution for washing and dried, the operations were alternately performed for 30 times in this way, finally washing was performed by using water for injection, drying was performed and the film was peeled off to obtain an implantable tissue adhesive film loading the ganglioside drug.

### Embodiment 9

In an aseptic bench, a culture dish with diameter of 12 cm was provided, a high-molecular material film with the same diameter was put therein (subjected to washing, sterilization and depyrogenation treatment), washing was performed by using water for injection and drying was performed; solution A of a material with a cationic group (1 mg/ml collagen, 0.1 mol/L acetic acid, 0.2 mol/L NaCl, pH=3.5) and solution B of a material with an anionic group (1 mg/ml hyaluronic acid, 0.15 mol/L NaCl, pH=6) were respectively prepared; the solution B was added with an antibacterial peptide (1.5 mg/ml); the solution was respectively filled into a high-pressure instantaneous coating device, firstly the solution A was instantaneously coated at high pressure into the culture dish, drying was performed, then the solution B was instantaneously coated at high pressure, washing was performed by using water for injection, the solution A and the solution B were alternately coated in this way repeatedly for 80 times; and finally the solution A was instantaneously coated at high pressure, washing was performed by using water for injection, drying was performed, the film was peeled off, an adhesive side of the film was washed by using water for injection, and dried to obtain an implantable tissue adhesive film loading the antibacterial peptide drug.

### Embodiment 10

In an aseptic bench, a culture dish with diameter of 12 cm was provided, a high-molecular material film with the same diameter was put therein (subjected to washing, sterilization and depyrogenation treatment), washing was performed by using water for injection and drying was performed; solution A of a material with a cationic group (1 mg/ml collagen, 0.1 mol/L acetic acid, 0.2 mol/L NaCl, pH=3.5) and solution B of a material with an anionic group (1 mg/ml hyaluronic acid, 0.15 mol/L NaCl, pH=6) were respectively prepared; the solution B was added with interleukin-2 (0.5 mg/ml); the solution was respectively filled into a high-pressure instantaneous coating device, firstly the solution A was instantaneously coated at high pressure into the culture dish, drying was performed, then the solution B was instantaneously coated at high pressure, washing was performed by using water for injection, the solution A and the solution B were alternately coated in this way repeatedly for 50 times; and finally the solution A was instantaneously coated at high pressure, washing was performed by using water for injection, drying was performed, the film was peeled off, an adhesive side of the film was washed by using water for injection, and dried to obtain an implantable tissue adhesive film loading the interleukin-2 drug.

### Embodiment 11

In an aseptic bench, a culture dish with diameter of 12 cm was provided, a high-molecular material film with the same diameter was put therein (subjected to washing, sterilization and depyrogenation treatment), washing was performed by using water for injection and drying was performed; solution A of a material with a cationic group (1 mg/ml collagen, 0.1 mol/L acetic acid, 0.2 mol/L NaCl, pH=3.5) and solution B of a material with an anionic group (1 mg/ml hyaluronic acid, 0.15 mol/L NaCl, pH=6) were respectively prepared; the solution A was added with an Afatinib cationic PEG nanometer microsphere (the concentration is 0.5 mg/ml), and uniform stirring with a targeting polypeptide (1*10⁻⁴ mg/ml) was performed, wherein an amino acid sequence of the targeting polypeptide was: arginine-glycine-aspartic acid; the solution was respectively filled into a high-pressure instantaneous coating device, firstly the solution A was instantaneously coated at high pressure into the culture dish, drying was performed, then the solution B was instantaneously coated at high pressure, washing was performed by using water for injection, the solution A and the solution B were alternately coated in this way repeatedly for 200 times; and finally the solution A was instantaneously coated at high pressure, washing was performed by using water for injection, drying was performed, the film was peeled off, an adhesive side of the film was washed by using water for injection, and dried to obtain an implantable tissue adhesive film loading the Afatinib drug.

### Embodiment 12

In an aseptic bench, a culture dish with diameter of 12 cm was provided, a high-molecular material film with the same diameter was put therein (subjected to washing, sterilization and depyrogenation treatment), washing was performed by using water for injection and drying was performed; solution A of a material with a cationic group (1 mg/ml collagen, 0.1 mol/L acetic acid, 0.2 mol/L NaCl, pH=3.5) and solution B of a material with an anionic group (1 mg/ml hyaluronic acid, 0.15 mol/L NaCl, pH=6) were respectively prepared; the solution A was added with an Imatinib cationic PEG nanometer microsphere (the concentration is 0.8 mg/ml), and uniform stirring with a targeting polypeptide (1*10⁻⁴ mg/ml) was performed, wherein an amino acid sequence of the targeting polypeptide was: arginine-glycine-aspartic acid; the solution was respectively filled into a high-pressure instantaneous coating device, firstly the solution A was instantaneously coated at high pressure into the culture dish, drying was performed, then the solution B was instantaneously coated at high pressure, washing was performed by using water for injection, the solution A and the solution B were alternately coated in this way repeatedly for 200 times; and finally the solution A was instantaneously coated at high pressure, washing was performed by using water for injection, drying was performed, the film was peeled off, an adhesive side of the film was washed by using water for injection, and dried to obtain an implantable tissue adhesive film loading the Imatinib drug.

### Embodiment 13

In an aseptic bench, a culture dish with diameter of 12 cm was provided, a high-molecular material film with the same diameter was put therein (subjected to washing, sterilization and depyrogenation treatment), washing was performed by using water for injection and drying was performed; solution A of a material with a cationic group (1 mg/ml collagen, 0.1 mol/L acetic acid, 0.2 mol/L NaCl, pH=3.5) and solution B of a material with an anionic group (1 mg/ml hyaluronic acid, 0.15 mol/L NaCl, pH=6) were respectively prepared; the solution B was added with an Axitinib anionic PEG nanometer microsphere (the concentration is 0.8 mg/ml), and uniform stirring with a targeting polypeptide (1*10⁻⁴ mg/ml) was performed, wherein an amino acid sequence of the targeting polypeptide was: arginine-glycine-aspartic acid; the solution was respectively filled into a high-pressure instantaneous coating device, firstly the solution A was instantaneously coated at high pressure into the culture dish, drying was performed, then the solution B was instantaneously coated at high pressure, washing was performed by using water for injection, the solution A and the solution B were alternately coated in this way repeatedly for 200 times; and finally the solution A was instantaneously coated at high pressure, washing was performed by using water for injection, drying was performed, the film was peeled off, an adhesive side of the film was washed by using water for injection, and dried to obtain an implantable tissue adhesive film loading the Axitinib drug.

### Embodiment 14

In an aseptic bench, a culture dish with diameter of 12 cm was provided, a high-molecular material film with the same diameter was put therein (subjected to washing, sterilization and depyrogenation treatment), washing was performed by using water for injection and drying was performed; solution A of a material with a cationic group (1 mg/ml collagen, 0.1 mol/L acetic acid, 0.2 mol/L NaCl, pH=3.5) and solution B of a material with an anionic group (1 mg/ml hyaluronic acid, 0.15 mol/L NaCl, pH=6) were respectively prepared; the solution A was added with a Ceritinib cationic PEG nanometer microsphere (the concentration is 0.8 mg/ml), and uniform stirring with a targeting polypeptide (1*10⁻⁴ mg/ml) was performed, wherein an amino acid sequence of the targeting polypeptide was: arginine-glycine-aspartic acid; the solution was respectively filled into a high-pressure instantaneous coating device, firstly the solution A was instantaneously coated at high pressure into the culture dish, drying was performed, then the solution B was instantaneously coated at high pressure, washing was performed by using water for injection, the solution A and the solution B were alternately coated in this way repeatedly for 200 times; and finally the solution A was instantaneously coated at high pressure, washing was performed by using water for injection, drying was performed, the film was peeled off, an adhesive side of the film was washed by using water for injection, and dried to obtain an implantable tissue adhesive film loading the Ceritinib drug.

### Embodiment 15

In an aseptic bench, a culture dish with diameter of 12 cm was provided, a high-molecular material film with the same diameter was put therein (subjected to washing, sterilization and depyrogenation treatment), washing was performed by using water for injection and drying was performed; solution A of a material with a cationic group (1 mg/ml collagen, 0.1 mol/L acetic acid, 0.2 mol/L NaCl, pH=3.5) and solution B of a material with an anionic group (1 mg/ml hyaluronic acid, 0.15 mol/L NaCl, pH=6) were respectively prepared; the solution B was added with fluorouracil (1.0 mg/ml); the solution was respectively filled into a high-pressure instantaneous coating device, firstly the solution A was instantaneously coated at high pressure into the culture dish, drying was performed, then the solution B was instantaneously coated at high pressure, washing was performed by using water for injection, the solution A and the solution B were alternately coated in this way repeatedly for 100 times; and finally the solution A was instantaneously coated at high pressure, washing was performed by using water for injection, drying was performed, the film was peeled off, an adhesive side of the film was washed by using water for injection, and dried to obtain an implantable tissue adhesive film loading the fluorouracil drug.

### Embodiment 16

In an aseptic bench, a high-molecular material plate was provided (subjected to washing, sterilization and depyrogenation treatment), and drying was performed; solution A of a material with a cationic group (2 mg/ml chitosan, 0.1 mol/L acetic acid, 0.2 mol/L NaCl, pH=4) and solution B of a material with an anionic group (2 mg/ml alginic acid with a molecular weight of 60000-80000, 0.15 mol/L NaCl, pH=6) were respectively prepared; the solution B was added with a Pertuzumab drug (20 ug/ml), and the plate was firstly immersed in the solution A for 20 min, taken out, put in washing solution for washing and dried; and then the plate was immersed in the solution B for 30 min, taken out, put in washing solution for washing and dried, the operations were alternately performed for 100 times in this way, finally washing was performed by using water for injection, drying was performed and the film was peeled off to obtain an implantable tissue adhesive film loading the Pertuzumab drug.

### Embodiment 17

In an aseptic bench, a culture dish with diameter of 12 cm was provided, a high-molecular material film with the same diameter was put therein (subjected to washing, sterilization and depyrogenation treatment), washing was performed by using water for injection and drying was performed; solution A of a material with a cationic group (1 mg/ml collagen, 0.1 mol/L acetic acid, 0.2 mol/L NaCl, pH=3.5) and solution B of a material with an anionic group (1 mg/ml hyaluronic acid, 0.15 mol/L NaCl, pH=6) were respectively prepared; the solution B was added with a Pertuzumab drug (20 ug/ml); the solution was respectively filled into a high-pressure instantaneous coating device, firstly the solution A was instantaneously coated at high pressure into the culture dish, drying was performed, then the solution B was instantaneously coated at high pressure, washing was performed by using water for injection, the solution A and the solution B were alternately coated in this way repeatedly for 100 times; and finally the solution A was instantaneously coated at high pressure, washing was performed by using water for injection, drying was performed, the film was peeled off, an adhesive side of the film was washed by using water for injection, and dried to obtain an implantable tissue adhesive film loading the Pertuzumab drug.

### Embodiment 18

In an aseptic bench, a culture dish with diameter of 12 cm was provided, a high-molecular material film with the same diameter was put therein (subjected to washing, sterilization and depyrogenation treatment), washing was performed by using water for injection and drying was performed; solution A of a material with a cationic group (1 mg/ml collagen, 0.1 mol/L acetic acid, 0.2 mol/L NaCl, pH=3.5) and solution B of a material with an anionic group (1 mg/ml hyaluronic acid, 0.15 mol/L NaCl, pH=6) were respectively prepared; the solution B was added with a Lidamycin (0.5 mg/ml, macromolecular protein antitumor antibiotics); the solution was respectively filled into a high-pressure instantaneous coating device, firstly the solution A was instantaneously coated at high pressure into the culture dish, drying was performed, then the solution B was instantaneously coated at high pressure, washing was performed by using water for injection, the solution A and the solution B were alternately coated in this way repeatedly for 80 times; and finally the solution A was instantaneously coated at high pressure, washing was performed by using water for injection, drying was performed, the film was peeled off, an adhesive side of the film was washed by using water for injection, and dried to obtain an implantable tissue adhesive film loading the Lidamycin drug.

### Embodiment 19

In an aseptic bench, a high-molecular material plate was provided (subjected to washing, sterilization and depyrogenation treatment), and drying was performed; solution A of a material with a cationic group (2 mg/ml chitosan, 0.1 mol/L acetic acid, 0.2 mol/L NaCl, pH=4) and solution B of a material with an anionic group (2 mg/ml hyaluronic acid, 0.15 mol/L NaCl, pH=6) were respectively prepared; the solution B was added with a vascular endothelial growth factor (VEGF) (0.5 mg/ml), and uniform stirring with a targeting polypeptide (1*10⁻⁴ mg/ml) was performed, wherein an amino acid sequence of the targeting polypeptide was: valine-glycine-valine-alanine-proline-glycine; the plate was firstly immersed in the solution A for 20 min, taken out, put in washing solution for washing and dried; and then the plate was immersed in the solution B for 30 min, taken out, put in washing solution for washing and dried, the operations were alternately performed for 80 times in this way, finally washing was performed by using water for injection, drying was performed and the film was peeled off to obtain a tissue adhesive film loading the vascular endothelial growth factor (VEGF) drug.

### Embodiment 20

In an aseptic bench, a high-molecular material plate was provided (subjected to washing, sterilization and depyrogenation treatment), and drying was performed; solution A of a material with a cationic group (2 mg/ml chitosan, 0.1 mol/L acetic acid, 0.2 mol/L NaCl, pH=4) and solution B of a material with an anionic group (2 mg/ml hyaluronic acid, 0.15 mol/L NaCl, pH=6) were respectively prepared; the solution B was added with an epidermal growth factor (EGF) (0.5 mg/ml), and uniform stirring was performed; the plate was firstly immersed in the solution A for 20 min, taken out, put in washing solution for washing and dried; and then the plate was immersed in the solution B for 30 min, taken out, put in washing solution for washing and dried, the operations were alternately performed for 100 times in this way, finally washing was performed by using water for injection, drying was performed and the film was peeled off to obtain a tissue adhesive film loading the epidermal growth factor (EGF) drug.

### Embodiment 21

In an aseptic bench, a culture dish with diameter of 12 cm was provided, a high-molecular material film with the same diameter was put therein (subjected to washing, sterilization and depyrogenation treatment), washing was performed by using water for injection and drying was performed; solution A of a material with a cationic group (1 mg/ml collagen, 0.1 mol/L acetic acid, 0.2 mol/L NaCl, pH=3.5) and solution B of a material with an anionic group (1 mg/ml hyaluronic acid, 0.15 mol/L NaCl, pH=6) were respectively prepared; the solution B was added with a nerve growth factor (NGF) (0.5 mg/ml); the solution was respectively filled into a high-pressure instantaneous coating device, firstly the solution A was instantaneously coated at high pressure into the culture dish, drying was performed, then the solution B was instantaneously coated at high pressure, washing was performed by using water for injection, the solution A and the solution B were alternately coated in this way repeatedly for 50 times; and finally the solution A was instantaneously coated at high pressure, washing was performed by using water for injection, drying was performed, the film was peeled off, an adhesive side of the film was washed by using water for injection, and dried to obtain an implantable tissue adhesive film loading the nerve growth factor (NGF) drug.

### Embodiment 22

In an aseptic bench, a culture dish with diameter of 12 cm was provided, a high-molecular material film with the same diameter was put therein (subjected to washing, sterilization and depyrogenation treatment), washing was performed by using water for injection and drying was performed; solution A of a material with a cationic group (1 mg/ml collagen, 0.1 mol/L acetic acid, 0.2 mol/L NaCl, pH=3.5) and solution B of a material with an anionic group (1 mg/ml hyaluronic acid, 0.15 mol/L NaCl, pH=6) were respectively prepared; the solution B was added with a fibroblast growth factor (FGF) (0.5 mg/ml); the solution was respectively filled into a high-pressure instantaneous coating device, firstly the solution A was instantaneously coated at high pressure into the culture dish, drying was performed, then the solution B was instantaneously coated at high pressure, washing was performed by using water for injection, the solution A and the solution B were alternately coated in this way repeatedly for 80 times; and finally the solution A was instantaneously coated at high pressure, washing was performed by using water for injection, drying was performed, the film was peeled off, an adhesive side of the film was washed by using water for injection, and dried to obtain a tissue adhesive film loading the fibroblast growth factor (FGF) drug.

### Embodiment 23

In an aseptic bench, a high-molecular material plate was provided (subjected to washing, sterilization and depyrogenation treatment), and drying was performed; solution A of a material with a cationic group (2 mg/ml chitosan, 0.1 mol/L acetic acid, 0.2 mol/L NaCl, pH=4) and solution B of a material with an anionic group (2 mg/ml alginic acid with a molecular weight of 60000-80000, 0.15 mol/L NaCl, pH=6) were respectively prepared; the solution B was added with a heparin drug (1.5 mg/ml); the plate was firstly immersed in the solution A for 20 min, taken out, put in washing solution for washing and dried; and then the plate was immersed in the solution B for 30 min, taken out, put in washing solution for washing and dried, the operations were alternately performed for 100 times in this way, finally washing was performed by using water for injection, drying was performed and the film was peeled off to obtain a tissue adhesive film loading the heparin drug.

### Embodiment 24

In an aseptic bench, a culture dish with diameter of 12 cm was provided, a high-molecular material film with the same diameter was put therein (subjected to washing, sterilization and depyrogenation treatment), washing was performed by using water for injection and drying was performed; solution A of a material with a cationic group (1 mg/ml collagen, 0.1 mol/L acetic acid, 0.2 mol/L NaCl, pH=3.5) and solution B of a material with an anionic group (1 mg/ml hyaluronic acid, 0.15 mol/L NaCl, pH=6) were respectively prepared; the solution B was added with a Lycium barbarum polysaccharide drug (1.0 mg/ml); the solution was respectively filled into a high-pressure instantaneous coating device, firstly the solution A was instantaneously coated at high pressure into the culture dish, drying was performed, then the solution B was instantaneously coated at high pressure, washing was performed by using water for injection, the solution A and the solution B were alternately coated in this way repeatedly for 100 times; and finally the solution A was instantaneously coated at high pressure, washing was performed by using water for injection, drying was performed, the film was peeled off, an adhesive side of the film was washed by using water for injection, and dried to obtain a tissue adhesive film loading the Lycium barbarum polysaccharide drug.

### Embodiment 25

In an aseptic bench, a culture dish with diameter of 12 cm was provided, a high-molecular material film with the same diameter was put therein (subjected to washing, sterilization and depyrogenation treatment), washing was performed by using water for injection and drying was performed; solution A of a material with a cationic group (1 mg/ml collagen, 0.1 mol/L acetic acid, 0.2 mol/L NaCl, pH=3.5) and solution B of a material with an anionic group (1 mg/ml hyaluronic acid, 0.15 mol/L NaCl, pH=6) were respectively prepared; the solution B was added with lentinan (0.5 mg/ml); the solution was respectively filled into a high-pressure instantaneous coating device, firstly the solution A was instantaneously coated at high pressure into the culture dish, drying was performed, then the solution B was instantaneously coated at high pressure, washing was performed by using water for injection, the solution A and the solution B were alternately coated in this way repeatedly for 80 times; and finally the solution A was instantaneously coated at high pressure, washing was performed by using water for injection, drying was performed, the film was peeled off, an adhesive side of the film was washed by using water for injection, and dried treatment was performed to obtain an implantable tissue adhesive film loading the lentinan drug.

### Embodiment 26

In an aseptic bench, a high-molecular material plate was provided (subjected to washing, sterilization and depyrogenation treatment), and drying was performed; solution A of a material with a cationic group (2 mg/ml chitosan, 0.1 mol/L acetic acid, 0.2 mol/L NaCl, pH=4) and solution B of a material with an anionic group (2 mg/ml hyaluronic acid, 0.15 mol/L NaCl, pH=6) were respectively prepared; the solution B was added with pachymaran (0.5 mg/ml), and uniform stirring with a targeting polypeptide (1*10⁻⁴ mg/ml) was performed, wherein an amino acid sequence of the targeting polypeptide was: valine-glycine-valine-alanine-proline-glycine; the plate was firstly immersed in the solution A for 20 min, taken out, put in washing solution for washing and dried; and then the plate was immersed in the solution B for 30 min, taken out, put in washing solution for washing and dried, the operations were alternately performed for 80 times in this way, finally washing was performed by using water for injection, drying was performed and the film was peeled off to obtain a tissue adhesive film loading the pachymaran drug.

### Embodiment 27

In an aseptic bench, a high-molecular material plate was provided (subjected to washing, sterilization and depyrogenation treatment), and drying was performed; solution A of a material with a cationic group (2 mg/ml chitosan, 0.1 mol/L acetic acid, 0.2 mol/L NaCl, pH=4) and solution B of a material with an anionic group (2 mg/ml hyaluronic acid, 0.15 mol/L NaCl, pH=6) were respectively prepared; the solution B was added with Polyporus polysaccharide (0.5 mg/ml), and uniform stirring was performed; the plate was firstly immersed in the solution A for 20 min, taken out, put in washing solution for washing and dried; and then the plate was immersed in the solution B for 30 min, taken out, put in washing solution for washing and dried, the operations were alternately performed for 100 times in this way, finally washing was performed by using water for injection, drying was performed and the film was peeled off to obtain a tissue adhesive film loading the Polyporus polysaccharide drug.

### Embodiment 28

The drug-loaded tissue adhesive film drug delivery systems obtained in embodiments 1-27 were subjected to biocompatibility evaluation detection, and showed excellent biocompatibility, specifically as follows:

1. Cytotoxicity Test:

Reference/technical standard: GB/T 16886.5-2003

Cell line: L-929 cells (mouse fibroblasts)

Culture solution: DMEM with 10% (v/v) calf serum

Blank control: the same-batch cell culture solution

Negative control: high density polyethylene (see GB/T16886 Cytotoxicity Test Standard)

Positive control: 5 g/L phenol solution

Extraction medium: the same-batch DMEM with no calf serum

Extraction time: 24 h

Test sample extraction ratio: 1 g/5 ml

Test method: extract test (MTT method)

At 27° C., 5% CO₂ blank control, negative control, positive control and test sample extract contacted with adherently grown cells, culture was performed for 72 h, then MTT solution was added and incubation was performed for 4 h. After resorption, DMSO was added, the absorbance of each group at a wavelength of 630 nm was measured through a spectrophotometer, and the relative proliferation rate of the cells was calculated.

Results: cytotoxicity of the test sample: level 0

Conclusion: according to GB/T 16886.5-2003, the test sample is not cytotoxic.

2. Intradermal Stimulation Test

Reference/technical standard: GB/T 16886. 10-2005

Test animal: healthy New Zealand rabbit

Extract medium: 0.9% sodium chloride injection

Test sample: material extract

Negative control: the same-batch extraction medium

Contact route: intradermal injection

Evaluation index: erythema and edema reaction degree at 24 h, 48 h and 72 h

Result: there is no erythema and edema reaction in the local and peripheral skin tissues at 24 h, 48 h, and 72 h after injection, and the skin reaction on the test side is not greater than the skin reaction on the control side.

Conclusion: according to GB/T 16886. 10-2005, the test sample has no intradermal stimulation.

3. Acute Systemic Toxicity Test

Reference/technical standard: GB/T 16886. 11-1997/ASTM F 750

Test animals: healthy mice

Extraction medium: 0.9% sodium chloride injection

Test sample: Material extract

Negative control: the same-batch extract medium

Contact route: tail vein injection

Evaluation index: general state of animals, toxicity performance and number of dead animals at 4 h, 24 h, 48 h and 72 h

Results: the response of the animals in the test sample group is not greater than that in the negative control group during the observation period of 72 h.

Conclusion: referring to GB/T 16886. 11-1997/ASTM F 750, the results of the acute systemic toxicity test of the test sample are in line with requirements.

4. Hemolysis Test

Reference/technical standard: GB/T 16886. 4-2003/GB/T 16175-1996

Test animals: healthy New Zealand rabbits

Diluted anticoagulant rabbit blood preparation: fresh anticoagulant rabbit blood+0.9% sodium chloride injection

Negative control: 0.9% sodium chloride injection

Positive control: distilled water

Contact route: tail vein injection

Test method: the test sample was immersed in 0.9% sodium chloride injection at a certain ratio and then incubated at 37° C. for 30 min in a water bath, and 0.2 ml of fresh anticoagulant rabbit blood was added, and the mixture was incubated at 37° C. for 60 min. Centrifugation was performed at 2500 rpm for 5 min, then the supernatant was taken, and the absorbance was measured at 545 nm by using an ultraviolet spectrophotometer to calculate the hemolysis rate.

Result: the hemolysis rate of the test sample is 0.2%.

Conclusion: according to GB/T 16886.4-2003, the hemolysis test results of the test samples meet the requirements on medical materials.

### Embodiment 29

Stability and Integrity Test:

In an aseptic bench, a high-molecular material plate was provided (subjected to washing, sterilization and depyrogenation treatment), and drying was performed; solution A of a material with a cationic group (2 mg/ml chitosan, 0.1 mol/L acetic acid, 0.2 mol/L NaCl, pH=4) and solution B of a material with an anionic group (2 mg/ml hyaluronic acid, 0.15 mol/L NaCl, pH=6) were respectively prepared; part of the solution B was taken and added with a small interfering nucleic acid drug (the same as embodiment 1, 10 ug/ml) to prepare solution C, the solution was respectively filled into a high-pressure instantaneous coating device, firstly the solution A was instantaneously coated at high pressure into the culture dish, and drying was performed to form a film; then the solution B was instantaneously coated at high pressure and washing was performed by using water for injection; then the solution A was instantaneously coated at high pressure, washing was performed by using water for injection, the solution A and the solution B were alternately coated in this way repeatedly for four times; then the solution A was instantaneously coated at high pressure and washing was performed by using water for injection; then the solution C was instantaneously coated at high pressure, washing was performed by using water for injection, drying was performed, the solution A and the solution C were alternatively coated in this way repeatedly for seven times, the film was peeled off, an adhesive side of the film was washed by using water for injection, and dried to obtain an implantable tissue adhesive film loading the small interfering nucleic acid drug.

The multilayer film prepared above was placed in 1M NaCl solution and was incubated for a certain period of time, and the resulting slow-release solution was firstly subjected to filtration treatment through an ultrafiltration centrifuge tube with a molecular weight cut-off of 30 KDa to remove macromolecular substances. Then, it was filtered through an ultrafiltration centrifuge tube with a molecular weight cut-off of 3 KDa to remove salt ions in the slow-release solution, i.e., desalting treatment was performed. Finally, the treated slow-release solution was subjected to gel-running treatment as sample solution in an electrophoresis tank. Polyacrylamide gel electrophoresis (PAGE) was used herein to verify the gene integrity and related stability of siRNA released from the multilayer film.

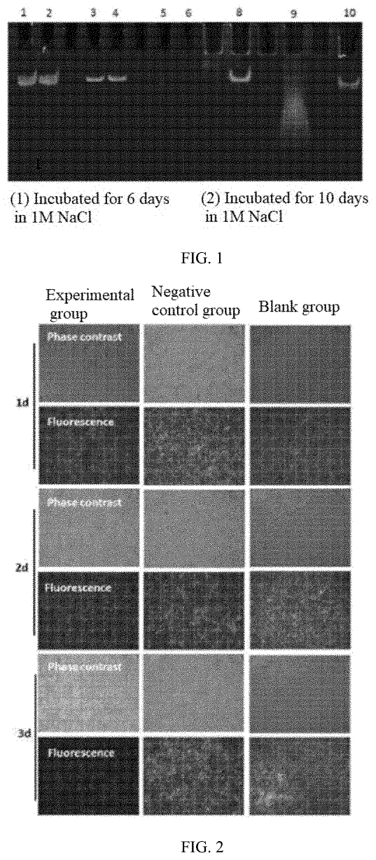

The gel electrophoresis experiment in FIG. 1 verified the stability and integrity of the slow-release siRNA sequence. The siRNA-loaded tissue adhesive film was incubated in 1M NaCl solution, and the film was gradually ruptured and dissolved. After 6 days, it was difficult to observe the molded product. From the gel electrophoresis experiment in FIG. 1, it can be seen that the dissociated siRNA could not be observed after 6 days of desalting, while the dissociated siRNA could be clearly seen after 10 days of desalting. Accordingly, it can be inferred that, in the process of gradual rupture and dissolution, the lysate is a combination of siRNA and anionic and cationic groups. With degrading of the anionic and cationic groups, siRNA is gradually dissociated and maintains the stability and integrity of the siRNA sequence.

### Embodiment 30

Cell Transfection Experiment:

In an aseptic bench, a high-molecular material plate was provided (subjected to washing, sterilization and depyrogenation treatment), and drying was performed; solution A of a material with a cationic group (2 mg/ml chitosan, 0.1 mol/L acetic acid, 0.2 mol/L NaCl, pH=4) and solution B of a material with an anionic group (2 mg/ml hyaluronic acid, 0.15 mol/L NaCl, pH=6) were respectively prepared; part of the solution B was taken and added with a small interfering nucleic acid drug (the same as embodiment 1, 10 ug/ml) to prepare solution C, the solution was respectively filled into a high-pressure instantaneous coating device, firstly the solution A was instantaneously coated at high pressure into the culture dish, and drying was performed to form a film; then the solution B was instantaneously coated at high pressure and washing was performed by using water for injection; then the solution A was instantaneously coated at high pressure, washing was performed by using water for injection, the solution A and the solution B were alternately coated in this way repeatedly for four times; then the solution A was instantaneously coated at high pressure and washing was performed by using water for injection; then the solution C was instantaneously coated at high pressure, washing was performed by using water for injection, drying was performed, the solution A and the solution C were alternatively coated in this way repeatedly for two times, the film was peeled off, an adhesive side of the film was washed by using water for injection, and dried to obtain an implantable tissue adhesive film loading the small interfering nucleic acid drug.

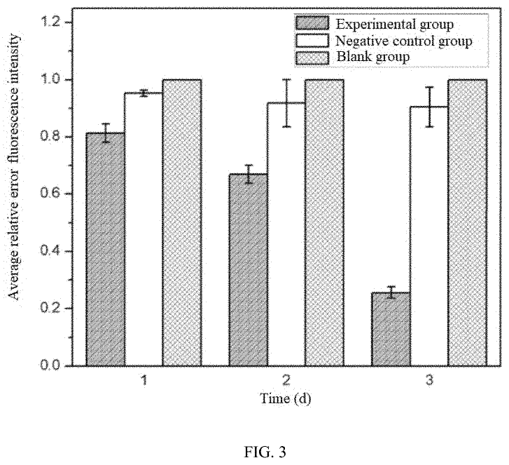

The tissue adhesive film prepared above was used as an experimental group, i.e., the experimental group was a film loading eGFP-siRNA which experienced alternate polyelectrolyte reaction twice, and the negative control group was a film loading common siRNA which experienced alternate polyelectrolyte reaction twice (the preparation method was the same as that of the experimental group, and only eGFP-siRNA was replaced with common siRNA). The experimental group and the negative control group were placed in a 6-well plate, respectively, and eGFP-HEK 293T cells were respectively inoculated into the 6-well plate (the cells were directly inoculated into wells in which no film was placed and were used as the blank control group). The changes in fluorescence intensity were observed on day 1, day 2 and day 3, and the results are shown in FIG. 2. In FIG. 3, the relative fluorescence intensities of the experimental group, the negative control group and the blank group are listed. It can be seen that the fluorescence intensity of the blank group and the negative control group is almost unchanged within 3 days, while the fluorescence intensity of the experimental group is significantly decreased with the increase of time within 3 days. Accordingly, it is verified that the tissue adhesive film loading eGFP siRNA can enable the fluorescence intensity of eGFP-HEK 293T cells to be reduced, and it indicates that eGFP siRNA has been transfected into the cells and has induced target gene silencing.

### Embodiment 31

Stability and Integrity Test:

In an aseptic bench, a culture dish with diameter of 12 cm was provided, a high-molecular material film with the same diameter was put therein (subjected to washing, sterilization and depyrogenation treatment), washing was performed by using water for injection and drying was performed; solution A of a material with a cationic group (1 mg/ml collagen, 0.1 mol/L acetic acid, 0.2 mol/L NaCl, pH=3.5) and solution B of a material with an anionic group (1 mg/ml hyaluronic acid, 0.15 mol/L NaCl, pH=6) were respectively prepared; the solution A was added with an Afatinib cationic PEG nanometer microsphere (the concentration is 0.5 mg/ml), and uniform stirring with a targeting polypeptide (1*10⁻⁴ mg/ml) was performed, wherein an amino acid sequence of the targeting polypeptide was: arginine-glycine-aspartic acid; the solution was respectively filled into a high-pressure instantaneous coating device, firstly the solution A was instantaneously coated at high pressure into the culture dish, drying was performed, then the solution B was instantaneously coated at high pressure, washing was performed by using water for injection, the solution A and the solution B were alternately coated in this way repeatedly for 200 times; and finally the solution A was instantaneously coated at high pressure, washing was performed by using water for injection, drying was performed, the film was peeled off, an adhesive side of the film was washed by using water for injection, and dried to obtain an implantable tissue adhesive film loading the Afatinib drug.

The multilayer film prepared above was placed in 1M NaCl solution and was incubated for a certain period of time, the obtained slow-release solution was purified, Afatinib could be obtained through separation, and the amount of Afatinib obtained through separation was not significantly reduced relative to the amount of Afatinib added during preparation. Accordingly, it can be seen that the stability and integrity of the drug are maintained.

### Embodiment 32

Cell Transfection Experiment:

In an aseptic bench, a high-molecular material plate was provided (subjected to washing, sterilization and depyrogenation treatment), and drying was performed; solution A of a material with a cationic group (2 mg/ml chitosan, 0.1 mol/L acetic acid, 0.2 mol/L NaCl, pH=4) and solution B of a material with an anionic group (2 mg/ml hyaluronic acid, 0.15 mol/L NaCl, pH=6) were respectively prepared; the solution B was added with a vascular endothelial growth factor (VEGF) (0.5 mg/ml), and uniform stirring with a targeting polypeptide (1*10⁻⁴ mg/ml) was performed, wherein an amino acid sequence of the targeting polypeptide was: valine-glycine-valine-alanine-proline-glycine; the plate was firstly immersed in the solution A for 20 min, taken out, put in washing solution for washing and dried; and then the plate was immersed in the solution B for 30 min, taken out, put in washing solution for washing and dried, the operations were alternately performed for 80 times in this way, finally washing was performed by using water for injection, drying was performed and the film was peeled off to obtain a tissue adhesive film loading the vascular endothelial growth factor (VEGF) drug.

The multilayer film prepared above was placed in 1M NaCl solution and was incubated for a certain period of time, the obtained slow-release solution was purified, the vascular endothelial growth factor could be obtained through separation, and as detected by using a VEGF detection kit, the amount of the VEGF obtained through separation was not significantly reduced relative to the amount of the VEGF added during preparation. Accordingly, it can be seen that the stability and integrity of the drug are maintained.

### Embodiment 33

In an aseptic bench, a high-molecular material plate was provided (subjected to washing, sterilization and depyrogenation treatment), and drying was performed; solution A of a material with a cationic group (2 mg/ml chitosan, 0.1 mol/L acetic acid, 0.2 mol/L NaCl, pH=4) and solution B of a material with an anionic group (2 mg/ml hyaluronic acid, 0.15 mol/L NaCl, pH=6) were respectively prepared; the solution B was added with Polyporus polysaccharide (0.5 mg/ml), and uniform stirring was performed; the plate was firstly immersed in the solution A for 20 min, taken out, put in washing solution for washing and dried; and then the plate was immersed in the solution B for 30 min, taken out, put in washing solution for washing and dried, the operations were alternately performed for 100 times in this way, finally washing was performed by using water for injection, drying was performed and the film was peeled off to obtain a tissue adhesive film loading the Polyporus polysaccharide drug.

The multilayer film prepared above was placed in 1M NaCl solution and was incubated for a certain period of time to obtain slow-release solution. By measuring the polysaccharide components in the slow-release solution, it can be found that the amount of the polysaccharide in the slow-release solution obtained through separation was not significantly reduced relative to the amount of the polysaccharide added during preparation. Accordingly, it can be seen that the stability and integrity of the drug are maintained.

To sum up, the present invention effectively overcomes various disadvantages in the prior art and thus has a great industrial utilization value.

The above-mentioned embodiments are just used for exemplarily describing the principle and effect of the present invention instead of limiting the present invention. One skilled in the art may make modifications or changes to the above-mentioned embodiments without departing from the spirit and scope of the present invention. Therefore, all equivalent modifications or changes made by those who have common knowledge in the art without departing from the spirit and technical thought disclosed by the present invention shall be still covered by the claims of the present invention.

1. A drug-loaded tissue adhesive film, comprising alternately superposed cationic layers and anionic layers, wherein at least one of the cationic layers and the anionic layers is a drug layer, or at least one of the cationic layers and the anionic layers contains a drug with charges.

2. The drug-loaded tissue adhesive film according to claim 1, characterized in that, when at least one of the cationic layers and the anionic layers is the drug layer, the cationic layer and/or the anionic layer used as the drug layer is the drug with charges, the cationic layer not used as the drug layer contains a carrier material with a cationic group, and the anionic layer not used as the drug layer contains a carrier material with an anionic group; and when at least one of the cationic layers and the anionic layers contains the drug with charges, the cationic layer contains a carrier material with a cationic group and the anionic layer contains a carrier material with an anionic group.

3. The drug-loaded tissue adhesive film according to claim 2, characterized in that the carrier material with a cationic group is one or a combination of an organic high-molecular polymer with a cationic group, a polysaccharide with a cationic group, a polypeptide with a cationic group, a protein with a cationic group, and a cationic liposome.

4. The drug-loaded tissue adhesive film according to claim 2, characterized in that the carrier material with an anionic group is one or a combination of an organic high-molecular polymer with an anionic group, a polysaccharide with an anionic group, a polypeptide with an anionic group, a protein with an anionic group, and an anionic liposome.

5. The drug-loaded tissue adhesive film according to claim 2, characterized in that the carrier material with a cationic group and the carrier material with an anionic group are biocompatible materials.

6. The drug-loaded tissue adhesive film according to claim 1, characterized in that the cationic layer presents positive charges on the whole and the anionic layer presents negative charges on the whole.

7. The drug-loaded tissue adhesive film according to claim 1, characterized in that the drug with charges is a drug complex.

8. A method for preparing the drug-loaded tissue adhesive film according to claim 1, comprising the following step: alternately depositing cationic layers and anionic layers on a substrate to prepare the tissue adhesive film.

9. Application of a tissue adhesive film to preparation of a drug carrier material, the tissue adhesive film comprises alternately superposed cationic layers and anionic layers.

10. The application according to claim 9, characterized in that at least one of the cationic layers and the anionic layers is a drug layer, or at least one of the cationic layers and the anionic layers is used for containing a drug with charges.
